(19) United States
(12) Patent Application Publication
(10) Pub. No.: US 2024/0296554 A1
(43) Pub. Date: Sep. 5, 2024

(54) METHOD AND SYSTEM FOR DATA BALANCING AND HAIR-LINE FRACTURE DETECTION

(71) Applicant: L&T TECHNOLOGY SERVICES LIMITED, Chennai (IN)

(72) Inventors: ARANGARAJAN PALANIAPPAN, Tiruchengode (IN); AKSHAYA BABU, kochi (IN); SWAROOPKUMAR MYSORE LOKESH, Mysore (IN)

(21) Appl. No.: 18/232,375

(22) PCT Filed: Apr. 8, 2022
(86) PCT No.: PCT/IB2022/053314

§ 371 Date: Aug. 10, 2023

(30) Foreign Application Priority Data
Jun. 23, 2021 (IN) 202141028234

(51) Int. Cl. G06T 7/00; G06V 10/764

(52) U.S. Cl. CPC G06T 7/0012; G06V 10/764; G06T 2207/10116; G06T 2207/30008

(57) ABSTRACT

The present disclosure describes a technique for balancing an imbalanced image dataset comprising fracture images and non-fracture images, where the fracture images are less than the non-fracture images. The balancing of the image dataset is performed by augmenting the fracture images. The technique of the present disclosure generates a classifier model based on the fracture and non-fracture images and further generates a generator model using the classifier model. The technique of the present disclosure augments the plurality of fracture images by applying the generated classifier and generator models. The technique of the present disclosure further involves iteratively performing generating of classifier and generator models; and augmenting the fracture images until the image dataset becomes a balanced dataset.

### TECHNICAL FIELD

The present disclosure generally relates to the field of data augmentation. Particularly, the present disclosure relates to a data balancing technique for detecting a hairline fracture in an object.

### BACKGROUND

Image processing is a technique to perform various operations on an image to extract useful information from the image. Nowadays image processing is widely used in medical image processing. One of the major applications of image processing is in fracture detection using X-ray images. A fracture may occur when an external force exercised upon a bone is more than the threshold force the bone can bear.

Minor cracks developed in human skeletal, or bones may be referred as hairline fractures or hairline cracks. Thus, in the field of medical radiology, a hairline fracture is a small crack within a bone or human skeletal. Hairline fractures usually develop gradually because of overuse or repetitive actions, as opposed to larger bone fractures or breaks that are mostly caused by acute traumas, such as falls, accidents, etc. An increase in either the duration or frequency of an activity can result in a hairline fracture.

Conventionally, expert radiologists place X-Ray films in front of a light source for detecting fractures. However, since the hairline fractures are minor in nature, they are hardly visible to the human eye using the conventional techniques. Hence, it is challenging for the radiologists to detect the hairline fracture using human eye.

Nowadays, various Machine Learning (ML) and deep learning techniques have been developed for detecting the hairline fractures. These techniques use deep learning models for detecting the hairline fractures. Initially the deep learning models are trained over a plurality of datasets. The accuracy of a deep learning model for detecting the hairline fractures depends on the type of dataset used (i.e., whether the dataset is a balanced dataset or an imbalanced dataset). The deep learning models trained over imbalanced datasets are less accurate compared to the deep learning models trained over balanced datasets. A balanced dataset is a dataset where the proportions of fracture data and non-fracture data used for training the deep learning models is almost the same. Existing deep learning techniques use imbalanced datasets for training the deep learning models because in the medical field it is difficult to find balanced datasets. Moreover, the cost associated with obtaining the balanced datasets is very high. Hence, the conventional deep learning models, which are trained over imbalanced datasets, are less accurate in detecting hairline fractures.

Further, detection of hairline fractures or cracks is equally important in engineering and industrial applications. Particularly, detection of hairline fractures or cracks in electrical/electronics, mechanical or civil structures/components/equipment/appliances is very important to ensure proper functioning or working of the structures/components/equipment/appliances. For example, hairline cracks are very common in buildings (particularly in plaster, which is prone to accidents if not timely repaired), nuts, bolts and other components installed in windmills and other structures that are hard to access, concrete foundations, airplane parts, ships, pressure vessels, pipelines, metal sheets, medical devices etc.

Thus, with the huge and rapidly growing demand of hairline fracture detection in the field of medical radiology and other industrial applications, there exists a need for further improvements in the existing techniques. Particularly, there exists a need for efficient techniques that can generate balanced datasets to be used for accurate detection of hairline fractures or cracks.

### SUMMARY

One or more shortcomings discussed above are overcome, and additional advantages are provided by the present disclosure. Additional features and advantages are realized through the techniques of the present disclosure. Other embodiments and aspects of the disclosure are described in detail herein and are considered a part of the disclosure.

An object of the present disclosure is to provide efficient techniques for balancing imbalanced datasets for training a deep learning model.

Another objective of the present disclosure is to provide efficient techniques for accurately detecting hairline fractures in an object.

Yet another objective of the present disclosure is to classify an input image of an object as a fracture image or a non-fracture image.

The above stated objects as well as other objects, features, and advantages of the present disclosure will become clear to those skilled in the art upon review of the following description, the attached drawings, and the appended claims.

In a non-limiting embodiment, an image augmentation method is disclosed. The method may comprise a step (a) receiving a plurality of fracture images and a plurality of non-fracture images, where the plurality of fracture images is less than the plurality of non-fracture images. The method may further comprise step (b) training a classifier to generate a classifier model; step (c) training a generator to generate a generator model; and step (d) augmenting the plurality of fracture images. The augmenting the plurality of fracture images may comprise iteratively processing each of the plurality of non-fracture images using the generator model for adding a plurality of corresponding fracture components into the plurality of non-fracture images for generating a plurality of synthetic fracture images corresponding to the plurality of non-fracture images. The augmenting the plurality of fracture images may further comprise iteratively processing each of the plurality of synthetic fracture images using the classifier model for generating a plurality of fracture scores corresponding to the plurality of synthetic fracture images. The augmenting the plurality of fracture images may further comprise selecting a predefined number of synthetic fracture images from the plurality of synthetic fracture images based on the plurality of fracture scores and generating a plurality of augmented fracture images by adding the predefined number of synthetic fracture images to the plurality of fracture images. The method may further comprise a step (e) iteratively performing steps (b)-(d) based on the plurality of augmented fracture images and the plurality of non-fracture images until the plurality of augmented fracture images becomes proportional to the plurality of non-fracture images.

In yet another non-limiting embodiment of the present disclosure, a method for detecting a hairline fracture in an object is described. The method may comprise the method as described in paragraphs for generating the plurality of augmented fracture images and detecting a hairline fracture in an object using the classifier by generating a classifier model based on the generated plurality of augmented fracture images and the plurality of non-fracture images.

In yet another non-limiting embodiment of the present disclosure, an image augmentation system is described. The image augmentation system may comprise a memory and at least one processor operatively coupled to the memory. The at least one processor may be configured to perform operations as described in paragraph [0013].

In yet another non-limiting embodiment of the present disclosure, a system for detecting a hairline fracture in an object is described. The system may comprise the at least one processor for generating the plurality of augmented fracture images. The at least one processor may be configured to detect a hairline fracture in an object using the classifier by generating a classifier model based on the generated plurality of augmented fracture images and the plurality of non-fracture images.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF DRAWINGS

Further aspects and advantages of the present disclosure will be readily understood from the following detailed description with reference to the accompanying drawings. Reference numerals have been used to refer to identical or functionally similar elements. The figures together with a detailed description below, are incorporated in and form part of the specification, and serve to further illustrate the embodiments and explain various principles and advantages, in accordance with the present disclosure wherein:

FIG. 1 shows an exemplary environment of a communication system 100 for image data augmentation for detecting a hair-line fracture in an object, in accordance with some embodiments of the present disclosure.

FIG. 2 shows a detailed block diagram 200 of the communication system 100 illustrated in FIG. 1, in accordance with some embodiments of the present disclosure.

FIG. 3 shows a process flow diagram for image data augmentation, in accordance with some embodiments of the present disclosure.

FIG. 4 shows a process flow diagram for training a generator, in accordance with some embodiments of the present disclosure.

FIGS. 5(a)-5(b) show flowcharts illustrating an image augmentation method 500, in accordance with some embodiments of the present disclosure.

FIG. 6 shows a flowchart illustrating a method 600 for training a classifier, in accordance with some embodiments of the present disclosure.

FIG. 7 shows a flowchart illustrating a method 700 for training a generator, in accordance with some embodiments of the present disclosure.

It should be appreciated by those skilled in the art that any block diagrams herein represent conceptual views of the illustrative systems embodying the principles of the present disclosure. Similarly, it will be appreciated that any flowcharts, flow diagrams, state transition diagrams, pseudo code, and the like represent various processes which may be substantially represented in computer readable medium and executed by a computer or processor, whether or not such computer or processor is explicitly shown.

### DETAILED DESCRIPTION

In the present document, the word “exemplary” is used herein to mean “serving as an example, instance, or illustration.” Any embodiment or implementation of the present disclosure described herein as “exemplary” is not necessarily to be construed as preferred or advantageous over other embodiments.

While the disclosure is susceptible to various modifications and alternative forms, specific embodiments thereof have been shown by way of example in the drawings and will be described in detail below. It should be understood, however, that it is not intended to limit the disclosure to the particular form disclosed, but on the contrary, the disclosure is to cover all modifications, equivalents, and alternatives falling within the spirit and the scope of the disclosure.

The terms “comprise(s)”, “comprising”, “include(s)”, or any other variations thereof, are intended to cover a non-exclusive inclusion, such that a setup, device, apparatus, system, or method that comprises a list of components or steps does not include only those components or steps but may include other components or steps not expressly listed or inherent to such setup or device or apparatus or system or method. In other words, one or more elements in a device or system or apparatus proceeded by “comprises . . . a” does not, without more constraints, preclude the existence of other elements or additional elements in the system.

The terms like “at least one” and “one or more” may be used interchangeably throughout the description. The terms like “a plurality of” and “multiple” may be used interchangeably throughout the description.

In the following detailed description of the embodiments of the disclosure, reference is made to the accompanying drawings that form a part hereof, and in which are shown by way of illustration of specific embodiments in which the disclosure may be practiced. These embodiments are described in sufficient detail to enable those skilled in the art to practice the disclosure, and it is to be understood that other embodiments may be utilized and that changes may be made without departing from the scope of the present disclosure. The following description is, therefore, not to be taken in a limiting sense. In the following description, well known functions or constructions are not described in detail since they would obscure the description with unnecessary detail.

Detection of hairline fractures from X-ray images using human eyes is difficult. Nowadays, deep learning models have been introduced to help the doctors or radiologists in detecting the hairline fractures without any difficulty. The deep learning models require training over a large number of datasets. Each dataset comprises a plurality of images having fracture (i.e., fracture images or minority class) and a plurality of images not having any fracture (i.e., normal images or non-fracture images or majority class). A well-balanced dataset (having a balance of minority class and majority class) is very important for creating a good prediction model. However, medical datasets are often imbalanced datasets, where one type of class labels are very rare. For example, in present scenario, the number of fracture images are extremely less compared to the number of non-fracture images in a dataset.

The imbalanced distribution of the fracture and non-fracture images pose a challenge for predictive modeling as most of deep learning algorithms used for classification are designed around the assumption of an equal number of examples for each type of images. Hence, the existing classification methods tend to perform poorly on minority class (i.e., fracture images). Thus, it is challenging to accurately detect hairline fracture using the existing classification methods.

The present disclosure overcome these and other problems by first balancing the dataset by augmenting the plurality of fracture images and then training a deep learning model based on the balanced dataset. Thus, the deep learning model can accurately detect hairline fractures as it is trained on the balanced dataset.

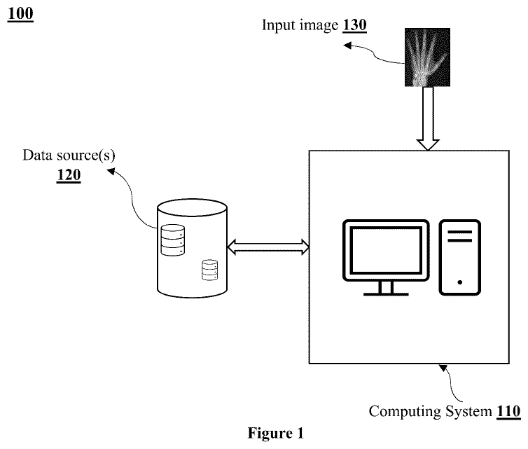

Referring now to FIG. 1, which illustrates a communication system 100 for image data augmentation for detecting a hair-line fracture in an object, in accordance with some embodiments of the present disclosure. The communication system 100 may comprise a computing system 110 (also referred as image augmentation system or hairline fracture detection system or system) which may be in communication with one or more data sources 120. Additionally, the computing system 110 may be in communication with other computing systems (not shown) or communication devices via at least one network (not shown).

The network may comprise a data network such as, but not restricted to, the Internet, Local Area Network (LAN), Wide Area Network (WAN), Metropolitan Area Network (MAN), etc. In certain embodiments, the network may include a wireless network, such as, but not restricted to, a cellular network and may employ various technologies including Enhanced Data rates for Global Evolution (EDGE), General Packet Radio Service (GPRS), Global System for Mobile Communications (GSM), Internet protocol Multimedia Subsystem (IMS), Universal Mobile Telecommunications System (UMTS) etc. In one embodiment, the network may include or otherwise cover networks or subnetworks, each of which may include, for example, a wired or wireless data pathway.

The one or more data sources 120 may comprise a plurality images of a plurality of objects. The plurality of images may comprise a plurality of fracture images and a plurality of normal images (i.e., non-fracture images). In one embodiment, the plurality images may be digital X-ray images. The plurality of images may be captured by any external device (not shown) using conventionally known X-ray imaging techniques and stored in the one or more data sources 120. In another embodiment, the plurality images may be non-X-ray images (i.e., normal photographs) captured by an image capturing device. Particularly, for internal fractures such as in human body parts, the plurality of images may be X-ray images and for fractures/cracks on surface, the plurality of images may be non-X-ray images. The plurality of images may be used for the purpose of training deep learning models.

In one non-limiting embodiment of the present disclosure, the computing system 110 may receive an input image 130 of an object. In one embodiment, the object may be a body part of a living being, or it may be any electrical/electronics, mechanical, or civil structure/component/equipment/appliance. The input image 130 may be captured by an external device and then transmitted to the computing system 130 or the computing system 110 may fetch the input image 130 from a memory of the external device. The computing system 110 may use deep learning models for classifying the input image 130 as a fracture image or a non-fracture image. It may be noted here that in one embodiment, the computing system 110 may be configured to receive a plurality of input images 130 of one or more objects.

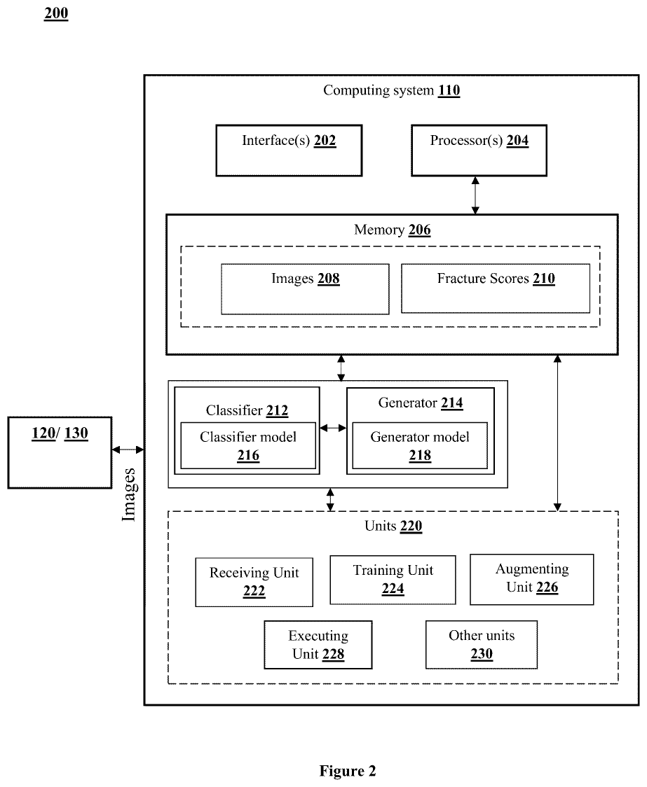

Now, FIG. 1 is explained in conjunction with FIG. 2, which is a detailed block diagram 200 of the communication system 100 for data augmentation for detecting a hair-line fracture in an object, in accordance with some embodiments of the present disclosure. According to an embodiment of the present disclosure, the computing system 110 may comprise one or more interfaces 202, at least one processor 204, a memory 206, a classifier 212, a generator 214, and various units 220. The units 220 may comprise a receiving unit 222, a training unit 224, an augmenting unit 226, a determining unit 228, and various other units 230. The various other units 230 may comprise a transmitting unit, a selecting unit, a generating unit, a comparing unit, and a tuning unit. In an embodiment, the units 222-230 may be dedicated hardware units capable of executing one or more instructions stored in the memory 206 for performing various operations of the computing system 110. In another embodiment, the units 222-230 may be software modules stored in the memory 206 which may be executed by the at least one processor 204 for performing the operations of the computing system 110. In one embodiment, some or all of the functionalities of the units 222-230 may be performed by the at least one processor 204. The classifier may be configured to generate a classifier model 216 and the generator may be configured to generate a generator model 218.

The interfaces 202 may include a variety of software and hardware interfaces, for example, a web interface, a graphical user interface, an input device-output device (I/O) interface, a network interface and the like. The I/O interfaces may allow the computing system 110 to interact with other computing systems directly or through other devices. The network interface may allow the computing system 110 to interact with one or more data sources 120 either directly or via a network.

The memory 206 may comprise various types of data. For example, the memory 206 may comprise one or more types of images 208 and fracture scores 210. The one or more type of images 208 may comprise a plurality of fracture images, a plurality of non-fracture images, a plurality of synthetic fracture (SF) images, one or more input images, a plurality of augmented fracture images. The memory 206 may further store one or more instructions executable by the at least one processor 204. The memory 206 may be communicatively coupled to the at least one processor 204. The memory 206 may include a Random-Access Memory (RAM) unit and/or a non-volatile memory unit such as a Read Only Memory (ROM), optical disc drive, magnetic disc drive, flash memory, Electrically Erasable Read Only Memory (EEPROM), a memory space on a server or cloud and so forth.

The at least one processor 204 may include, but not restricted to, a general-purpose processor, a Field Programmable Gate Array (FPGA), an Application Specific Integrated Circuit (ASIC), a Digital Signal Processor (DSP), microprocessors, microcomputers, micro-controllers, central processing units, state machines, logic circuitries, and/or any devices that manipulate signals based on operational instructions. A processor may also be implemented as a combination of computing devices, e.g., a combination of a DSP and a microprocessor, a plurality of microprocessors, one or more microprocessors in conjunction with a DSP core, or any other such configuration.

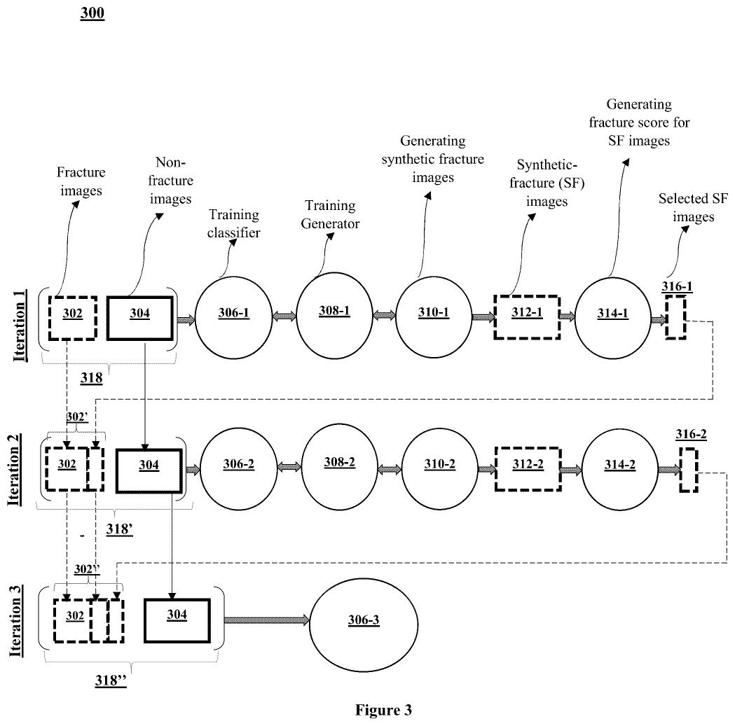

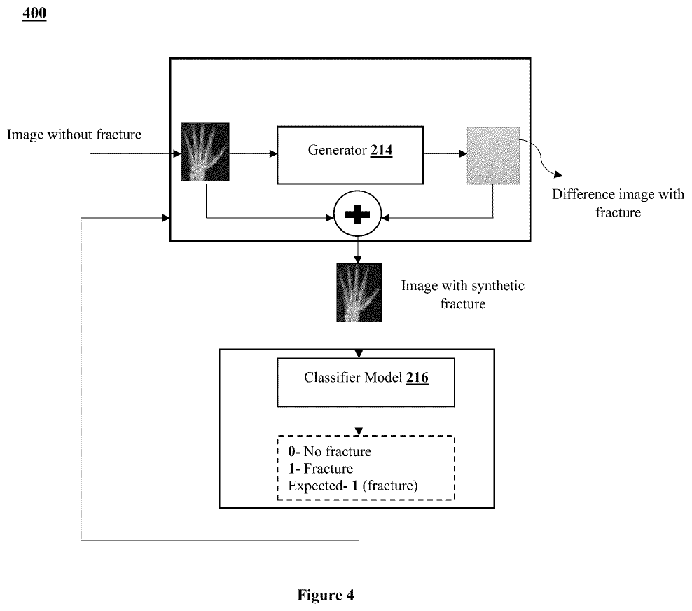

In the forthcoming paragraphs, the present disclosure is explained by means of FIGS. 3 and 4 in conjunction with FIGS. 1 and 2 (discussed above).

Referring now to FIG. 3, which illustrates a process flow diagram 300 depicting one or more operations performed by the computing system 110 for augmenting the plurality of fracture images, in accordance with some embodiments of the present disclosure. The various operations of FIG. 3 may be performed by the at least one processor 204. However, the present disclosure is not limited thereto, and the various operations may also be performed by one or more of the various units 216 of FIG. 2. As shown in FIG. 3, augmenting of the plurality fracture images may be performed iteratively. For example, in a first iteration, the at least one processor 204 may be configured to generate synthetic fracture images and a fix number of the generated synthetic fracture images (i.e., best synthetic fracture images) may be selected and added in the plurality of fracture images to obtain augmented fracture images. The at least one processor 204 may keep augmenting the plurality of fracture images iteratively until the number of fracture images becomes approximately proportional to the number of non-fracture images.

### Iteration 1:

In one non-limiting embodiment of the present disclosure, the at least one processor 204 may be configured to receive a plurality of images 318 from the one or more data sources 120. The plurality of images 318 may comprise a plurality of fracture images 302 and a plurality of non-fracture images 304. The plurality of fracture images 302 are very less than the plurality of non-fracture images 304. The at least one processor 204 may be configured to augment the plurality of fracture images 302 to make them proportional to the plurality of non-fracture images 304 by performing following operations.

### Training the Classifier:

In operation 306-1, the at least one processor 204 may be configured to train a classifier based on the plurality of images 318 to generate a classifier model. The plurality of images 318 may comprise a plurality of fracture images 302 and a plurality of non-fracture images 304. The classifier is a deep neural network, which is a computational graph comprising a plurality of nodes. Some nodes of the plurality of nodes are functions without parameters and other nodes are functions with parameters, where the parameters can be tuned. The parameters may comprise filters (F), weights (W), and biases (B). The nodes without parameters may include non-linear functions and operations. The non-linear functions may comprise “ReLU” or “Sigmoid”, which non-linearly maps an input to an output. The operations may comprise element-wise matrix addition, concatenation, etc. (concatenation and addition of two inputs). The nodes with parameters may include 2D Convolution (i.e., convolution of the input with a tune-able filter (F)), matrix multiplication (i.e., multiplication of the input with a tune-able weight matrix (W) followed by addition with a bias vector (B)). The values of the parameters are set to some default values before training the classifier.

In one non-limiting embodiment of the present disclosure, the structure of the computational graph is generally as follows but may be modified as per requirement. A sequence of convolution nodes is interspersed with non-linear functions and element-wise additions or concatenations with the outputs of previous convolution nodes, followed by one or more matrix multiplication nodes interspersed with non-linear functions and ending with a matrix multiplication node whose output is a vector of length C (where C is a number of categories), followed by a final non-linear function which maps the output to the range [0-1]. The i^{th} element of the output is interpreted as the classifier's confidence that the input image is of the i^{th} category. In the present disclosure, the output of the classifier may be considered as a fracture score having a value in the range of [0-1]. The actual fracture score corresponding to a fracture image may be considered as 1 and the actual fracture score corresponding to a non-fracture image may be considered as 0. The fracture score output from the classifier indicates the classifier's capability of classifying an input image as a fracture image or a non-fracture image.

The training of the classifier is performed in operation 306-1 of FIG. 3. In one non-limiting embodiment of the present disclosure, the at least one processor 204 may be configured to randomly select one image from the plurality of images 318. The selected image may be either a fracture image or a non-fracture image. The at least one processor 204 may be configured to process the selected image using the classifier to generate a fracture score for the selected image. The generated fracture score may be in the range of [0-1], where the score 1 corresponds to a fracture image and the score 0 corresponds to a non-fracture image.

It may be noted here that the plurality of images 318 are labelled images and each of the plurality of images has an associated actual fracture score i.e., actual fracture scores corresponding to the plurality of images 310 are already known to the at least one processor 204.

In one non-limiting embodiment, the generated fracture score may be indicative of the classifier's capability of classifying the selected image as a fracture image or a non-fracture image.

The at least one processor 204 may be configured to compare the generated fracture score of the selected image with the actual fracture score associated with the selected image and may determine a difference between the generated fracture score and the actual fracture score. The difference may also be indicative of the accuracy/correctness of the classifier while determining whether the selected image is the fracture image or the non-fracture image. For an ideal classifier, the difference should be zero. If the difference is large, it means that the classifier is unable to correctly classify the selected image as the fracture image or the non-fracture image. Thus, it is desired to minimize the difference to the best possible extent.

In one non-limiting embodiment of the present disclosure, the at least one processor 204 may be configured to tune one or more parameters of the classifier so as to minimize the difference between the generated fracture score and the actual fracture score. Tuning the one or more parameters may comprise choosing a set of optimal parameters for the classifier which minimizes the difference between the generated fracture score and the actual fracture score.

In one non-limiting embodiment of the present disclosure, the difference between the generated fracture score and the actual fracture score is also known as the degree of error, which is computed using a loss function such as, but not limited to, cross entropy, mean squared error. The parameters of the classifier may then be updated using techniques such as, but not limited to, gradient descent technique such that the degree of error is minimized.

In one non-limiting embodiment of the present disclosure, the at least one processor 204 may be configured to iteratively perform the classifier training operation 306-1 described in paragraphs [0048-0053] until a set of optimal classifier parameters is achieved.

In one non-limiting embodiment of the present disclosure, the at least one processor 204 may determine that the classifier parameters are optimal when it is not possible to minimize the difference or the degree of error beyond a limit. For example, when the value of difference remains constant/invariant for a predefined number of iterations (i.e., the classifier performance stops improving), the at least one processor 204 may determine that the current classifier parameters are optimal parameters for the available dataset of fracture images and non-fracture images.

In another non-limiting embodiment of the present disclosure, the at least one processor 204 may train the classifier for a predetermined time duration and may select the classifier parameters at the end of the time duration as the optimal classifier parameters. In yet another embodiment of the present disclosure, the at least one processor 204 may train the classifier for a predetermined number of iterations and may select the classifier parameters at the end of last iteration as the optimal classifier parameters.

The at least one processor 204 may then generate a classifier model whose parameters are set to the determined optimal classifier parameters. The classifier model generated in operation 306-1 is a decent classifier model for classifying an input image as the fracture image or the non-fracture image but not the best model.

### Training the Generator:

Once the classifier model is generated, the classifier parameters are frozen (i.e., fixed) and the at least one processor 204 may be configured to train the generator for generating a generator model. A fully trained generator is configured to generate a plurality of synthetic fracture images corresponding to the plurality of non-fracture images. Similar to the classifier, the generator is also a deep neural network, which is a computational graph comprising a plurality of nodes. Some nodes of the plurality of nodes are functions without parameters and other nodes are functions with parameters, where the parameters can be tuned. The parameters may comprise filters (F), weights (W), and biases (B) having default values, which may be tuned while training the generator.

The structure of the computational graph is generally as follows but may be modified as per the requirement. A sequence of convolution nodes is interspersed with non-linear functions and element-wise additions or concatenation with the outputs of previous convolution nodes, followed by one or more matrix multiplication nodes interspersed with non-linear functions and ending with a matrix multiplication node whose output is a vector of length B (where B is called the bottleneck dimension). This is followed by a sequence of either 2D transposed convolutions (unlike normal convolutions, transposed convolutions generate outputs of higher dimension than inputs) or 2D convolution nodes followed by parameter-less up-sampling nodes (which interpolate the input to generate an output of higher dimension), interspersed with non-linear functions. The final output is of the same dimension as the input image.

The training of the generator is performed in operation 308-1 of FIG. 3. The detailed operation of training the generator is described in FIG. 4.

Referring now to FIG. 4, which illustrates a process 400 for training the generator over the plurality of non-fracture images 304. Initially, the at least one processor 204 may be configured to randomly select one image from the plurality of non-fracture images 304. The at least one processor 204 may then be configured to process the selected image using the generator for generating a synthetic fracture image corresponding to the selected image by adding a fracture component into the selected image. The actual score corresponding to the generated synthetic fracture image is 1. Synthetic fracture image is defined as an artificially generated fracture version of the selected non-fracture image.

For generating the synthetic fracture image, the at least one processor 204 may be configured to process the selected image using the generator to generate a difference image corresponding to the selected image. The difference image may be defined as an image having only fracture component. In one embodiment, the difference image may be considered as a matrix/array of pixels with same width and height as the selected image. It may be noted that each image of the plurality of fracture and non-fracture images may be a digital X-ray image or a non-X-ray image. The at least one processor 204 may then combine (pixel-wise addition) the selected image with the generated difference image to generate the synthetic fracture image. However, since the generator is not a trained generator, it may not be capable of accurately generating the synthetic fracture image. Thus, in worst case, the generated synthetic fracture image may be a non-fracture image. The classifier model generated in operation 306-1 is therefore used to check whether the generated synthetic fracture image is a fracture image or not.

The at least one processor 204 may be configured to process the synthetic fracture image corresponding to the selected image using the classifier model for generating a fracture score. This fracture score may be indicative of the generator's capability of generating the synthetic fracture image. Consider an example, where the generated fracture score is 0.1 while the expected fracture score was 1. This means that the generator is not enough capable to accurately generate the synthetic fracture image corresponding to the selected non-fracture image.

The at least one processor 204 may be configured to compare the generated fracture score of the selected non-fracture image with the actual fracture score (which is 1) and may determine a difference between the generated fracture score and the actual fracture score. The difference may be indicative of the accuracy/correctness of the generator while generating the synthetic fracture image. For an ideal generator, the difference should be zero. If the difference is large, it means that the generator is unable to accurately generate the synthetic fracture image. Thus, it is desired to minimize the difference to the best possible extent.

In one non-limiting embodiment of the present disclosure, the at least one processor 204 may be configured to tune one or more parameters of the generator to minimize the difference between the generated fracture score and the actual fracture score. Tuning the one or more parameters may comprise choosing a set of optimal parameters for the generator which minimizes the difference between the generated fracture score and the actual fracture score.

In one non-limiting embodiment of the present disclosure, the difference between the generated fracture score and the actual fracture score is also known as the degree of error, which is computed using a loss function such as, but not limited to, cross entropy, mean squared error. The parameters of the generator may then be updated using techniques such as, but not limited to, gradient descent technique such that the degree of error is minimized.

In one non-limiting embodiment of the present disclosure, the at least one processor 204 may be configured to iteratively perform the generator training operation 308-1 described in paragraphs [0060-0066] until a set of optimal generator parameters is achieved/obtained.

In one non-limiting embodiment of the present disclosure, the at least one processor 204 may determine that the generator parameters are optimal when it is not possible to minimize the difference or the degree of error beyond a limit. For example, when the value of difference remains constant/invariant for a predefined number of iterations (i.e., the generator performance stops improving), the at least one processor 204 may determine that the current generator parameters are optimal generator parameters for the available dataset of fracture images and non-fracture images.

In another non-limiting embodiment of the present disclosure, the at least one processor 204 may train the generator for a predetermined time duration and may select the generator parameters at the end of the time duration as the optimal generator parameters. In yet another embodiment of the present disclosure, the at least one processor 204 may train the generator for a predetermined number of iterations and may select the parameters at the end of last iteration as the optimal generator parameters.

The at least one processor 204 may then generate a generator model whose parameters are set to the determined optimal generator parameters. The generator model generated in operation 308-1 is a satisfactory generator model for generating synthetic fracture images but not the best model.

In another embodiment of the present disclosure, the output of the classifier may be a vector y of length three. A first element of y may indicate the classifier's confidence that the selected image is non-fracture image, a second element of y may indicate the classifier's confidence that the selected image is a fracture image, and a third element of y may indicate the classifier's confidence that the selected image is a synthetic fracture image.

### Augmenting the Plurality of Fracture Images:

Once the classifier model and the generator model are generated, the at least one processor 204 may be configured to augment the plurality of fracture images 302 to make them proportional to the plurality of non-fracture images 304.

In operation 310-1, the at least one processor 204 may be configured to iteratively process each of the plurality of non-fracture images 304 using the generator model for adding a plurality of corresponding fracture components into the plurality of non-fracture images. By adding the plurality of corresponding fracture components into the plurality of non-fracture images, a plurality of synthetic fracture images 312-1 corresponding to the plurality of non-fracture images may be generated. For example, the at least one processor 204 may be configured to process the plurality of non-fracture images 304 using the generator model to generate a plurality of difference images corresponding to the plurality of non-fracture images using the technique described in paragraph [0062]. The at least one processor 204 may then combine the plurality of non-fracture images 304 with the corresponding generated difference images to generate the plurality of synthetic fracture images 312-1.

In one non-limiting embodiment of the present disclosure, the at least one processor 204 may be configured to process each of the plurality of synthetic fracture images 312-1 using the classifier model to generate a plurality of fracture scores corresponding to the plurality of synthetic fracture images 312-1 (operation 314-1).

In one non-limiting embodiment of the present disclosure, the at least one processor 204 may be configured to select a predefined number of synthetic fracture images 316-1 from the plurality of synthetic fracture images 312-1 based on the plurality of fracture scores. For selecting the predefined number of synthetic fracture images 316-1, the at least one processor 204 may be configured to first sort the plurality of synthetic fracture images 312-1 in descending order of fracture scores and then may select the predefined number of synthetic fracture images 316-1 starting from a synthetic fracture image having highest fracture score. In another embodiment, the at least one processor 204 may be configured to select a predefined percentage of synthetic fracture images from the plurality of synthetic fracture images 312-1 based on the fracture scores. For example, the at least one processor 204 may be configured to select top x % images from the list of the synthetic fracture images 312-1 arranged in descending order of the fracture scores, where x may have any value in the range of [0-100].

In an embodiment, the number of images selected based on the percentage may be equal to the predefined number of synthetic fracture images 316-1. In yet another non-limiting embodiment of the present disclosure, the at least one processor 204 may be configured to select synthetic fracture images from the plurality of synthetic fracture images 312-1 based on a predefined threshold value. For example, the at least one processor 204 may select only those synthetic fracture images for which the fracture score is above the predefined threshold value.

In one non-limiting embodiment of the present disclosure, the at least one processor 204 may be configured to generate a plurality of augmented fracture images 302′ by adding the predefined number of synthetic fracture images 316-1 to the plurality of fracture images 302. After generation of the plurality of augmented fracture images 302′, the first iteration ends.

### Iteration 2 . . . N

At the end of the first iteration, the plurality of augmented fracture images 302′ are generated. As can be seen from FIG. 3, the plurality of augmented fracture images 302′ are more in number than the plurality of fracture images 302. Hence, the plurality of augmented images 318′ at the end of first iteration will be more balanced in comparison to the plurality of original images 318. However, the plurality of augmented images 318′ may not be fully balanced. Thus, to further balance the plurality of augmented images 318′ the operations as described in iteration 1 may be repeated using input images as the plurality of augmented fracture images 302′ and the plurality of non-fracture images 304 until the plurality of images 318′ becomes balanced (i.e., until the number of fracture images in the plurality of augmented images 318′ becomes approximately proportional to the number of fracture images in the plurality of augmented images 318′).

For example, at the end of iteration 2, the plurality of augmented fracture images 302″ are generated which are proportional to the plurality of non-fracture images 304 and the augmenting process stops after second iteration. It may be noted that the at least one processor 204 may be configured to perform the process of iteration 1 up to N iterations (where N is an integer number) until fracture and non-fracture images become balanced.

It may be noted here that the number of fracture images in subsequent iterations is increasing (i.e., fracture and non-fracture images are gradually balancing). Thus, a classifier model and a generator model generated in a subsequent iteration will be more accurate than the previous iteration.

### Detecting Hairline Fracture:

In one non-limiting embodiment, when the plurality of images 318″ are balanced after N iterations (N=2 in the illustrative example of FIG. 3), in operation 306-3, the at least one processor 204 may be configured to train the classifier to generate a classifier model using input data as the plurality of augmented fracture images 302″ and the plurality of non-fracture images 304. Since the input data to the classifier is balanced data, the generated classifier model will be the best classifier model. The best classifier model is hereinafter referred as a final classifier model. The final classifier model may then be used for accurately detecting hairline fracture in an object.

In one non-limiting embodiment of the present disclosure, the at least one processor may receive an input image 130 of an object for detecting fracture. The at least one processor 204 may be configured to process the input image 130 using the final classifier to generate a fracture score corresponding to the input image 130. The at least one processor 204 may then classify the input image as having a fracture (i.e., a fracture image) or as not having a fracture (i.e., non-fracture image) based on the generated fracture score. For example, if the generated score is 1, the input image is classified as a fracture image and if the generated score is 0, the input image is classified as non-fracture image. In one embodiment, a range of fracture score may be defined for classifying the input image. For example, if the generated score is in the range of [0.8-1], the input image is a fracture image and if the generated score is in the range of [0-0.2], the input image is a non-fracture image.

In one non-limiting embodiment of the present disclosure, the at least one processor 204 may be configured to display the fracture score and the classification result (i.e., whether the input image is a fracture image or a non-fracture image) corresponding to the input image on a display of the computing system 110. In another embodiment of the present disclosure, the at least one processor may be configured to transmit the fracture score and the classification result to a mobile device associated with an intended user.

Thus, the present disclosure provides efficient and cost effective techniques for quickly balancing an imbalanced image dataset and then generating the final classifier model. Since, the final classifier model is generated on balanced dataset, the final classifier model is more accurate in classifying an input image. Therefore, the present disclosure provides efficient techniques for accurately detecting hairline fractures in an object. Further, since the techniques may automatically classify an input image, there is no need to depend on specialist/doctor. Hence, time and cost both are saved, and user experience is enhanced.

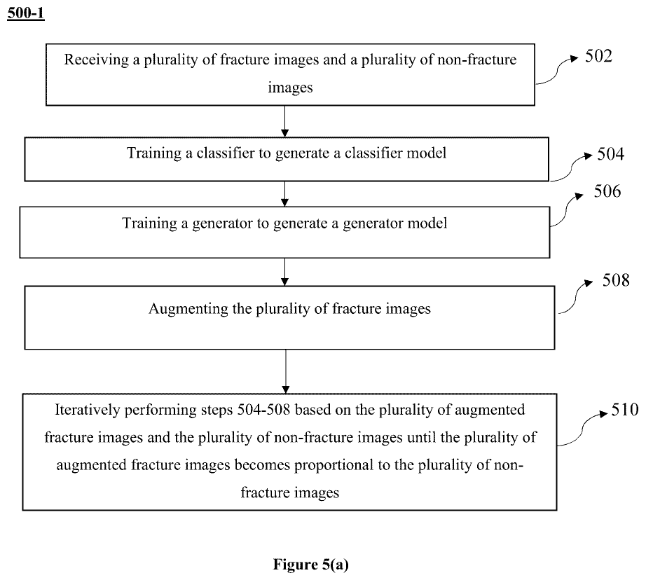

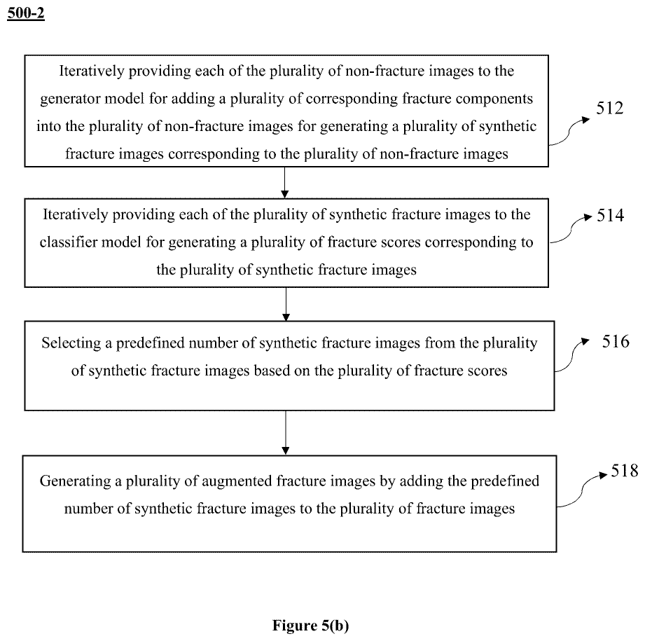

Referring now to FIGS. 5(a)-5(b) flowcharts are described illustrating exemplary image augmentation methods 500-1, 500-2 (collectively referred as 500), according to an embodiment of the present disclosure. The method 500 is merely provided for exemplary purposes, and embodiments are intended to include or otherwise cover any methods or procedures for evaluating one or more functionalities of an application.

The method 500-1 may include, at block 502, receiving a plurality of fracture images and a plurality of non-fracture images. The plurality of fracture images may be less than the plurality of non-fracture images. For example, the at least one processor 204 may be configured to receive the plurality of fracture images and the plurality of non-fracture images. The operations of block 402 may also be performed by the receiving unit 222 of FIG. 2.

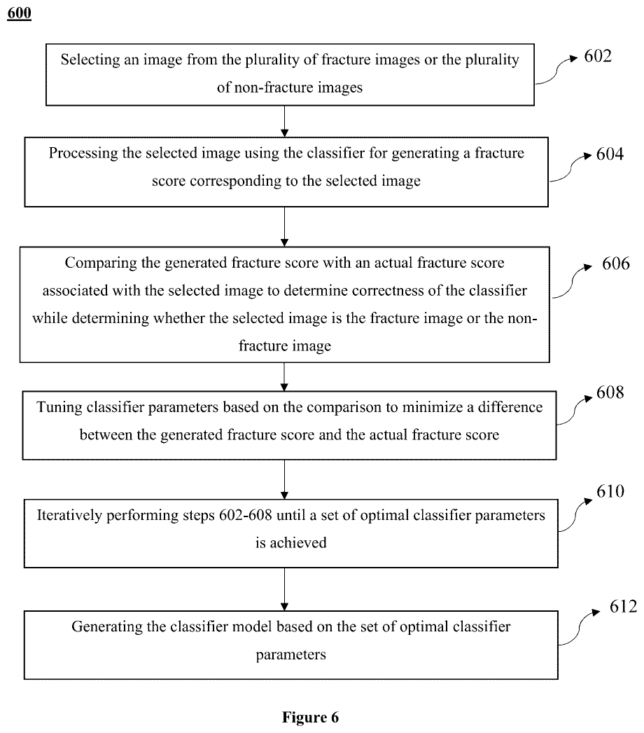

At block 504, the method 500-1 may include training a classifier to generate a classifier model. For example, the at least one processor 204 may be configured to train the classifier to generate the classifier model. The operations of block 504 may also be performed by the training unit 224 of FIG. 2. Detailed explanation of the training of the classifier is provided in FIG. 6.

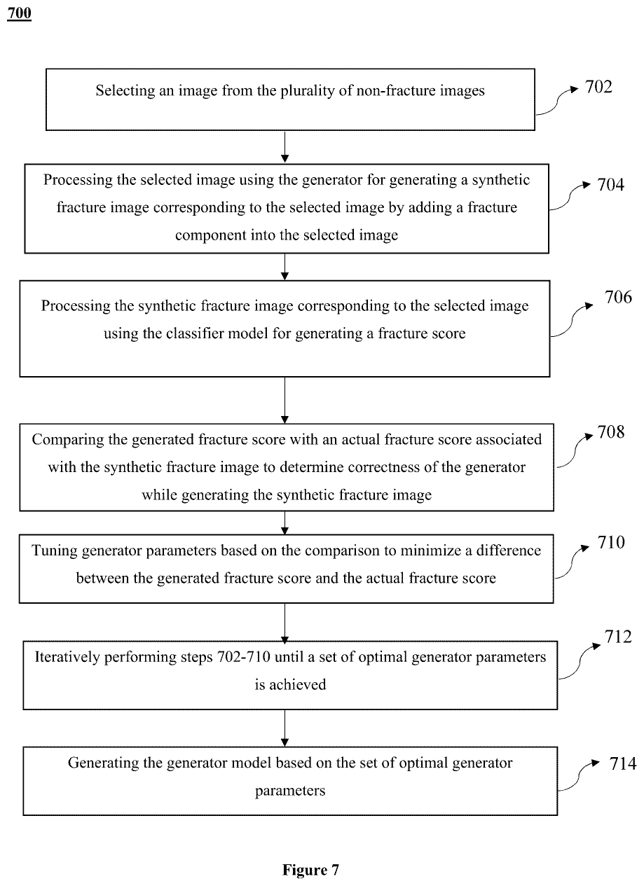

At block 506, the method 500-1 may include training a generator to generate a generator model. For example, the at least one processor 204 may be configured to train the generator to generate the generator model. The operations of block 506 may also be performed by the training unit 224 of FIG. 2. Detailed explanation of the training of the generator is provided in FIG. 7.

At block 508, the method 500-1 may include augmenting the plurality of fracture images to generate a plurality of augmented fracture images. For example, the at least one processor 204 may be configured to augment the plurality of fracture images to generate the plurality of augmented fracture images. The operations of block 508 may also be performed by the augmenting unit 226 of FIG. 2. The detailed explanation block 508 is provided in FIG. 5(b).

Referring now to FIG. 5(b) a flow chart is described, which illustrates a method 500-2 for augmenting the plurality of fracture images, according to an embodiment of the present disclosure. The various method steps described in FIG. 5(b) may be performed by the augmenting unit 226.

The method 500-2 may include, at block 512, iteratively processing each of the plurality of non-fracture images using the generator model for adding a plurality of corresponding fracture components into the plurality of non-fracture images for generating a plurality of synthetic fracture images corresponding to the plurality of non-fracture images. For example, the at least one processor 204 may be configured to iteratively process each of the plurality of non-fracture images using the generator model for adding the plurality of corresponding fracture components into the plurality of non-fracture images for generating the plurality of synthetic fracture images corresponding to the plurality of non-fracture images.

At block 514, the method 500-2 may include, iteratively processing each of the plurality of synthetic fracture images using the classifier model for generating a plurality of fracture scores corresponding to the plurality of synthetic fracture images. For example, the at least one processor 204 may be configured to iteratively process each of the plurality of synthetic fracture images using the classifier model for generating the plurality of fracture scores corresponding to the plurality of synthetic fracture images.

At block 516, the method 500-4 may include, selecting a predefined number of synthetic fracture images from the plurality of synthetic fracture images based on the plurality of fracture scores. For example, the at least one processor 204 may be configured to select the predefined number of synthetic fracture images from the plurality of synthetic fracture images based on the plurality of fracture scores.

At block 518, the method 500-4 may include, generating a plurality of augmented fracture images by adding the predefined number of synthetic fracture images to the plurality of fracture images. For example, the at least one processor 204 may be configured to generate the plurality of augmented fracture images by adding the predefined number of synthetic fracture images to the plurality of fracture images. In an embodiment, the various method steps described in FIG. 5(b) may be performed by the augmenting unit 226.

Coming back to FIG. 5(a), at block 510, the method 500-1 may include iteratively performing method steps 502-508 based on the plurality of augmented fracture images and the plurality of non-fracture images until the plurality of augmented fracture images becomes proportional to the plurality of non-fracture images. For example, the at least one processor 204 may be configured to iteratively perform the method steps 502-508 based on the plurality of augmented fracture images and the plurality of non-fracture images until the plurality of augmented fracture images becomes proportional to the plurality of non-fracture images. The operations of block 510 may also be performed by the executing unit 228 of FIG. 2.

In one non-limiting embodiment of the present disclosure, the method 500 may include generating a final classifier model using input data as the plurality of augmented fracture images 302″ and the plurality of non-fracture images 304 and detect a hairline fracture in an input image of the object using the final classifier model. For example, the at least one processor 204 may be configured to detect a hairline fracture in the input image of the object using the classifier by generating the final classifier model based on the generated plurality of augmented fracture images 302″ and the plurality of non-fracture images 304.

Referring now to FIG. 6 a flowchart is described illustrating an exemplary method 600 of training the classifier to generate the classifier model, according to an embodiment of the present disclosure. The method 600 is merely provided for exemplary purposes, and embodiments are intended to include or otherwise cover any methods or procedures for evaluating one or more functionalities of an application.

The method 600 may include, at block 602, selecting an image from the plurality of fracture images or the plurality of non-fracture images. For example, the at least one processor 204 may be configured to select the image from the plurality of fracture images or the plurality of non-fracture images.

At block 604, the method 600 may include, processing the selected image using the classifier for generating a fracture score corresponding to the selected image. The fracture score may be indicative of the classifier capability of determining whether the selected image is a fracture image or a non-fracture image. For example, the at least one processor 204 may be configured to process the selected image using the classifier for generating the fracture score corresponding to the selected image.

At block 606, the method 600 may include, comparing the generated fracture score with an actual fracture score associated with the selected image to determine correctness of the classifier while determining whether the selected image is the fracture image or the non-fracture image. For example, the at least one processor 204 may be configured to compare the generated fracture score with an actual fracture score associated with the selected image to determine correctness of the classifier while determining whether the selected image is the fracture image or the non-fracture image.

At block 608, the method 600 may include, tuning classifier parameters based on the comparison to minimize a difference between the generated fracture score and the actual fracture score. For example, the at least one processor 204 may be configured to tune classifier parameters based on the comparison to minimize the difference between the generated fracture score and the actual fracture score.

At block 610, the method 600 may include, iteratively performing steps 602-608 until a set of optimal classifier parameters is achieved. For example, the at least one processor 204 may be configured to iteratively perform the method steps 602-608 until a set of optimal classifier parameters is achieved.

At block 612, the method 600 may include, generating the classifier model based on the set of optimal classifier parameters. For example, the at least one processor 204 may be configured to generate the classifier model based on the set of optimal classifier parameters.

Referring now to FIG. 7 a flowchart is described illustrating an exemplary method 700 of training the generator to generate the generator model, according to an embodiment of the present disclosure. The method 700 is merely provided for exemplary purposes, and embodiments are intended to include or otherwise cover any methods or procedures for evaluating one or more functionalities of an application.

The method 700 may include, at block 702, selecting an image from the plurality of non-fracture images. For example, the at least one processor 204 may be configured to select the image from the plurality of non-fracture images.

At block 704, the method 700 may include, processing the selected image using the generator for generating a synthetic fracture image corresponding to the selected image by adding a fracture component into the selected image. For example, the at least one processor 204 may be configured to process the selected image using the generator for generating a synthetic fracture image corresponding to the selected image by adding the fracture component into the selected image.

At block 706, the method 700 may include, processing the synthetic fracture image corresponding to the selected image using the classifier model for generating a fracture score. The fracture score may be indicative of the generator capability of generating the synthetic fracture image. For example, the at least one processor 204 may be configured to process the synthetic fracture image corresponding to the selected image using the classifier model for generating the fracture score.

At block 708, the method 700 may include, comparing the generated fracture score with an actual fracture score associated with the synthetic fracture image to determine correctness of the generator while generating the synthetic fracture image. For example, the at least one processor 204 may be configured to compare the generated fracture score with the actual fracture score associated with the synthetic fracture image to determine correctness of the generator while generating the synthetic fracture image.

At block 710, the method 700 may include, tuning generator parameters based on the comparison to minimize a difference between the generated fracture score and the actual fracture score. For example, the at least one processor 204 may be configured to tune the generator parameters based on the comparison to minimize the difference between the generated fracture score and the actual fracture score.

At block 712, the method 700 may include, iteratively performing the method steps 702-710 until a set of optimal generator parameters is achieved. For example, the at least one processor 204 may be configured to iteratively performing the method steps 702-710 until the set of optimal generator parameters is achieved.

At block 714, the method 700 may include, generating the generator model based on the set of optimal generator parameters. For example, the at least one processor 204 may be configured to generating the generator model based on the set of optimal generator parameters.

The above methods 500, 600, 700 may be described in the general context of computer executable instructions. Generally, computer executable instructions can include routines, programs, objects, components, data structures, procedures, modules, and functions, which perform specific functions or implement specific abstract data types.

The order in which the various operations of the method are described is not intended to be construed as a limitation, and any number of the described method blocks can be combined in any order to implement the method. Additionally, individual blocks may be deleted from the methods without departing from the spirit and scope of the subject matter described herein. Furthermore, the methods can be implemented in any suitable hardware, software, firmware, or combination thereof.

It may be noted here that the subject matter of some or all embodiments described with reference to FIGS. 1-4 may be relevant for the methods and the same is not repeated for the sake of brevity. Further, it may be noted that some of the Figures and their corresponding description may have been described in the context of detecting hairline fractures in human body parts. However, the present disclosure is not limited thereto and is equally applicable to various electrical/electronics, mechanical, or civil structures/components/equipment/appliances etc. Particularly, the techniques of the present disclosure may be used to detect any type of defect (such as crack or fracture) in any electrical/electronics, mechanical, or civil structures/components/equipment/appliances.

Furthermore, one or more computer-readable storage media may be utilized in implementing embodiments consistent with the present disclosure. A computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., non-transitory. Examples include Random Access Memory (RAM), Read-Only Memory (ROM), volatile memory, nonvolatile memory, hard drives, Compact Disc (CD) ROMs, Digital Video Disc (DVDs), flash drives, disks, and any other known physical storage media.

Certain aspects may comprise a computer program product for performing the operations presented herein. For example, such a computer program product may comprise a computer readable media having instructions stored (and/or encoded) thereon, the instructions being executable by one or more processors to perform the operations described herein. For certain aspects, the computer program product may include packaging material.

Various components, modules, or units are described in this disclosure to emphasize functional aspects of devices configured to perform the disclosed techniques, but do not necessarily require realization by different hardware units. Rather, as described above, various units may be combined in a hardware unit or provided by a collection of interoperative hardware units, including one or more processors as described above, in conjunction with suitable software and/or firmware.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based here on. Accordingly, the embodiments of the present invention are intended to be illustrative, but not limiting, of the scope of the invention, which is set forth in the appended claims.

We claim:

1. An image augmentation method comprising:
(a) receiving a plurality of fracture images and a plurality of non-fracture images, wherein the plurality of fracture images is less than the plurality of non-fracture images;
(b) training a classifier to generate a classifier model;
(c) training a generator to generate a generator model;
(d) augmenting the plurality of fracture images by:
iteratively processing each of the plurality of non-fracture images using the generator model for adding a plurality of corresponding fracture components into the plurality of non-fracture images for generating a plurality of synthetic fracture images corresponding to the plurality of non-fracture images;
iteratively processing each of the plurality of synthetic fracture images using the classifier model for generating a plurality of fracture scores corresponding to the plurality of synthetic fracture images;
selecting a predefined number of synthetic fracture images from the plurality of synthetic fracture images based on the plurality of fracture scores; and
generating a plurality of augmented fracture images by adding the predefined number of synthetic fracture images to the plurality of fracture images; and
(e) iteratively performing steps (b)-(d) based on the plurality of augmented fracture images and the plurality of non-fracture images until the plurality of augmented fracture images becomes proportional to the plurality of non-fracture images.

2. The method as claimed in claim 1, wherein training a classifier to generate a classifier model comprises:
(b1) selecting an image from the plurality of fracture images or the plurality of non-fracture images;
(b2) processing the selected image using the classifier for generating a fracture score corresponding to the selected image, wherein the fracture score is indicative of the classifier capability of determining whether the selected image is a fracture image or a non-fracture image;
(b3) comparing the generated fracture score with an actual fracture score associated with the selected image to determine correctness of the classifier while determining whether the selected image is the fracture image or the non-fracture image;
(b4) tuning classifier parameters based on the comparison to minimize a difference between the generated fracture score and the actual fracture score;
(b5) iteratively performing steps (b1)-(b4) until a set of optimal classifier parameters is achieved; and
(b6) generating the classifier model based on the set of optimal classifier parameters.

3. The method as claimed in claim 1, wherein training a generator to generate a generator model comprises:
(c1) selecting an image from the plurality of non-fracture images;
(c2) processing the selected image using the generator for generating a synthetic fracture image corresponding to the selected image by adding a fracture component into the selected image;
(c3) processing the synthetic fracture image corresponding to the selected image using the classifier model for generating a fracture score, wherein the fracture score is indicative of the generator capability of generating the synthetic fracture image;
(c4) comparing the generated fracture score with an actual fracture score associated with the synthetic fracture image to determine correctness of the generator while generating the synthetic fracture image;
(c5) tuning generator parameters based on the comparison to minimize a difference between the generated fracture score and the actual fracture score;
(c6) iteratively performing steps (c1)-(c5) until a set of optimal generator parameters is achieved; and
(c7) generating the generator model based on the set of optimal generator parameters.

4. The method as claimed in claim 1, wherein the plurality of fracture images and the plurality of non-fracture images are X-ray images or non-X-ray images.

5. A method for detecting a hairline fracture in an object, the method comprising:
generating a plurality of augmented fracture images by:
(a) receiving a plurality of fracture images and a plurality of non-fracture images, wherein the plurality of fracture images is less than the plurality of non-fracture images;
(b) training a classifier to generate a classifier model;
(c) training a generator to generate a generator model;
(d) augmenting the plurality of fracture images by:
iteratively processing each of the plurality of non-fracture images using the generator model for adding a plurality of corresponding fracture components into the plurality of non-fracture images for generating a plurality of synthetic fracture images corresponding to the plurality of non-fracture images;
iteratively processing each of the plurality of synthetic fracture images using the classifier model for generating a plurality of fracture scores corresponding to the plurality of synthetic fracture images;
selecting a predefined number of synthetic fracture images from the plurality of synthetic fracture images based on the plurality of fracture scores; and
generating a plurality of augmented fracture images by adding the predefined number of synthetic fracture images to the plurality of fracture images; and
(e) iteratively performing steps (b)-(d) based on the plurality of augmented fracture images and the plurality of non-fracture images until the plurality of augmented fracture images becomes proportional to the plurality of non-fracture images; and
detecting a hairline fracture in an object using the classifier based on the generated plurality of augmented fracture images and the plurality of non-fracture images.

6. An image augmentation system comprising:
a memory;
at least one processor operatively coupled to the memory and configured to:
(a) receive a plurality of fracture images and a plurality of non-fracture images, wherein the plurality of fracture images is less than the plurality of non-fracture images;
(b) train a classifier to generate a classifier model;
(c) train a generator to generate a generator model;
(d) augment the plurality of fracture images by:
iteratively providing each of the plurality of non-fracture images to the generator model for adding a plurality of corresponding fracture components into the plurality of non-fracture images for generating a plurality of synthetic fracture images corresponding to the plurality of non-fracture images;
iteratively providing each of the plurality of synthetic fracture images to the classifier model for generating a plurality of fracture scores corresponding to the plurality of synthetic fracture images;
selecting a predefined number of synthetic fracture images from the plurality of synthetic fracture images based on the plurality of fracture scores; and
generating a plurality of augmented fracture images by adding the predefined number of synthetic fracture images to the plurality of fracture images; and
(e) iteratively perform steps (b)-(d) based on the plurality of augmented fracture images and the plurality of non-fracture images until the plurality of augmented fracture images becomes proportional to the plurality of non-fracture images.

7. The system as claimed in claim 6, wherein the at least one processor is configured to train a classifier to generate a classifier model by:
(b1) selecting an image from the plurality of fracture images or the plurality of non-fracture images;
(b2) processing the selected image using the classifier for generating a fracture score corresponding to the selected image, wherein the fracture score is indicative of the classifier capability of determining whether the selected image is a fracture image or a non-fracture image;
(b3) comparing the generated fracture score with an actual fracture score associated with the selected image to determine correctness of the classifier while determining whether the selected image is the fracture image or the non-fracture image;
(b4) tuning classifier parameters based on the comparison to minimize a difference between the generated fracture score and the actual fracture score;
(b5) iteratively performing steps (b1)-(b4) until a set of optimal classifier parameters is achieved; and
(b6) generating the classifier model based on the set of optimal classifier parameters.

8. The system as claimed in claim 6, wherein the at least processor is configured to train a generator to generate a generator model by:
(c1) selecting an image from the plurality of non-fracture images;
(c2) processing the selected image using the generator for generating a synthetic fracture image corresponding to the selected image by adding a fracture component into the selected image;
(c3) processing the synthetic fracture image corresponding to the selected image using the classifier model for generating a fracture score, wherein the fracture score is indicative of the generator capability of generating the synthetic fracture image;
(c4) comparing the generated fracture score with an actual fracture score associated with the synthetic fracture image to determine correctness of the generator while generating the synthetic fracture image;
(c5) tuning generator parameters based on the comparison to minimize a difference between the generated fracture score and the actual fracture score;
(c6) iteratively performing steps (c1)-(c5) until a set of optimal generator parameters is achieved; and
(c7) generating the generator model based on the set of optimal generator parameters.

9. The system as claimed in claim 6, wherein the plurality of fracture images and the plurality of non-fracture images are X-ray images or non-X-ray images.

10. A system for detecting a hairline fracture in an object, the system comprising:
a memory;
at least one processor operatively coupled to the memory and configured to:
(a) receive a plurality of fracture images and a plurality of non-fracture images, wherein the plurality of fracture images is less than the plurality of non-fracture images;
(b) train a classifier to generate a classifier model;
(c) train a generator to generate a generator model;
(d) augment the plurality of fracture images by:
iteratively providing each of the plurality of non-fracture images to the generator model for adding a plurality of corresponding fracture components into the plurality of non-fracture images for generating a plurality of synthetic fracture images corresponding to the plurality of non-fracture images;
iteratively providing each of the plurality of synthetic fracture images to the classifier model for generating a plurality of fracture scores corresponding to the plurality of synthetic fracture images;
selecting a predefined number of synthetic fracture images from the plurality of synthetic fracture images based on the plurality of fracture scores; and
generating a plurality of augmented fracture images by adding the predefined number of synthetic fracture images to the plurality of fracture images; and
(e) iteratively perform steps (b)-(d) based on the plurality of augmented fracture images and the plurality of non-fracture images until the plurality of augmented fracture images becomes proportional to the plurality of non-fracture images, wherein the at least one processor is configured to detect a hairline fracture in an object using the classifier based on the generated plurality of augmented fracture images and the plurality of non-fracture images.
